(19) United States
(12) Patent Application Publication
(10) Pub. No.: US 2023/0338697 A1
(43) Pub. Date: Oct. 26, 2023

(54) Automatic CPAP Humidifier Water Chamber Replenishing and Sanitizing System

(71) Applicant: Juan Sebastian Cardenas, Roanoke, VA (US)

(72) Inventor: Juan Sebastian Cardenas, Roanoke, VA (US)

(21) Appl. No.: 18/115,006

(22) Filed: Feb. 28, 2023

Related U.S. Application Data
(60) Provisional application No. 63/333,863, filed on Apr. 22, 2022.

(51) Int. Cl. A61M 16/16; A61M 16/00

(52) U.S. Cl. CPC A61M 16/165; A61M 16/024; A61M 2205/3327

(57) ABSTRACT

The present invention discloses a novel Automatic CPAP humidifier water chamber refilling and sanitizing system wherein contrary to the traditional approach of standard water chambers, which dry out and run out of water relatively quickly, the claimed assembly provides a constant and on-demand supply of fresh, clean and sanitized water to the CPAP water chamber; and keeps the CPAP water chamber constantly full and prevents the user from feeling discomfort associated with a dry water chamber. The proposed assembly can hold around 3 Liters of water and can supply the CPAP water chamber for many days depending on the CPAP user humidifier settings, for an estimated 7 to 10 days.

### BACKGROUND

### Field of the Invention

The present invention relates to an automatic system for replenishing water in a continuous positive airway pressure (CPAP) machine's humidifier water chamber. More specifically, the invention relates to an automatic CPAP water chamber replenishing system that replenishes water in the CPAP machine's water chamber during sleep without the need for user intervention.

### Description of the Related Art

Sleep Apnea Syndrome is a disorder characterized by cessation of breathing and frequent awakenings during sleep. One class of sleep apnea is obstructive sleep apnea, which is characterized by the obstruction of the person's upper airway resulting in interference with breathing during sleep in order to treat obstructive sleep apnea, Continuous Positive Airway Pressure (CPAP) devices have been developed to deliver air under constant positive pressure to the nasal passages during sleep. These devices are frequently successful in treating sleep deprivation due to obstructive sleep apnea.

In order to prevent drying of the breathing passages during the administration of CPAP, it is desirable to humidify the air supplied to the person using such devices This may be accomplished by providing a humidifier which is essentially a bottle containing a quantity of water over which the air under pressure passes before being delivered to the user. The user partially fills the bottle with water, and the air is circulated within the bottle to pass over the surface of the water and absorb moisture therefrom.

One of the challenges of using a CPAP machine is that the water in the water chamber can run out during the night. This can be a problem because it can cause discomfort or irritation to the nasal passages and throat. It can also disrupt sleep and cause the user to wake up and have to refill the water chamber.

There are multiple solutions that have been presented in prior art. However, these solutions are limited and restricted to their conventional architecture and have considerable shortcomings which adversely affect the convenience with which they can be used. The prior systems have certain limitations including the design of assemblies which make them not suitable for every type of user. Moreover, the ease-of-use ability of these assemblies is also questionable. The current invention also discusses an advancement in an automatic CPAP water chamber replenishing system that solves the problem of the water in the water chamber running out during the night. The automatic CPAP water chamber replenishing system is designed to automatically replenish the water in the water chamber during sleep without the need for user intervention.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present: invention and is not intended to be a full description. A full appreciation of the various aspects of the invention can be gained by taking the entire specification, claims, drawings, and abstract as a whole.

The primary desirable object of the present invention is to provide a novel and improved form of automatic CPAP water chamber replenishing system which aims to provide enhanced utility and convenience to the user.

The primary Objective of this invention is to provide an automatic CPAP water chamber replenishing system that includes a water reservoir connected to the CPAP machine's humidifier water chamber. The water reservoir is filled with distilled water before use and currently is being made to hold at least 3 Liters of water. The system also includes two external water level sensors that are connected to the water chamber of the CPAP machine. The sensors are designed to detect when the water level in the water chamber is low and when the water is high. Once the sensor detects that the water level in the water chamber is low, the automatic CPAP water chamber replenishing system is activated. The system is designed to release a predetermined amount of water from the water reservoir into the water chamber of the CPAP machine. The system is also designed to monitor the water level in the water chamber and repeat the replenishment process as needed throughout the night. The secondary sensor also has the functionality to stop the water pump if the water reaches it maximum level.

Another objective of the invention is to provide an easy-to-use device that provides convenience and comfort to the user and saves the user's effort and time.

It is a further objective of the invention to provide a device that unlike the prior art doesn't need the user's intervention to refill the water once the chamber is emptied.

It is also the objective of the invention to provide an automatic CPAP water chamber replenishing system designed to be user-friendly, easy to use, and easy to clean.

It is a further object of the present invention to provide a new and improved form of assembly that is of durable and reliable construction.

It is also the objective of the system to provide a universal assembly that works well with a wide range of CPAP machines.

Still, yet another object of the present invention is to provide a new and improved automatic CPAP humidifier water chamber replenishing and sanitizing system, that provides in the apparatuses and methods of the prior art some of the advantages thereof, while simultaneously overcoming some of the disadvantages normally associated therewith.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 depicts the side view of the assembly in accordance with the embodiment of the invention.

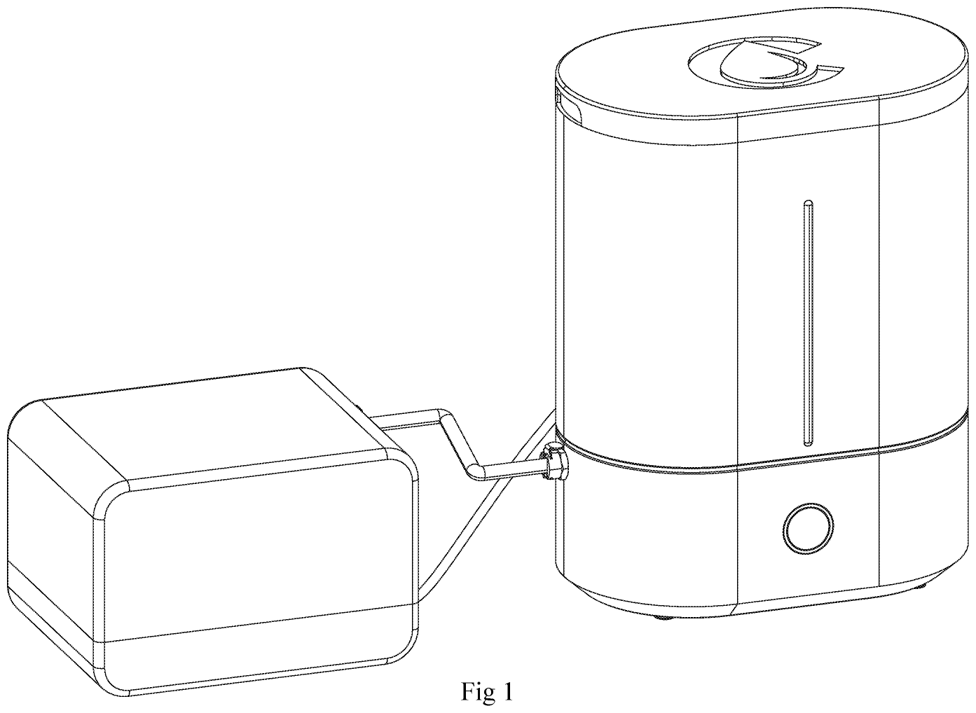

FIG. 2 depicts the front view of the assembly in accordance with the embodiment of the invention.

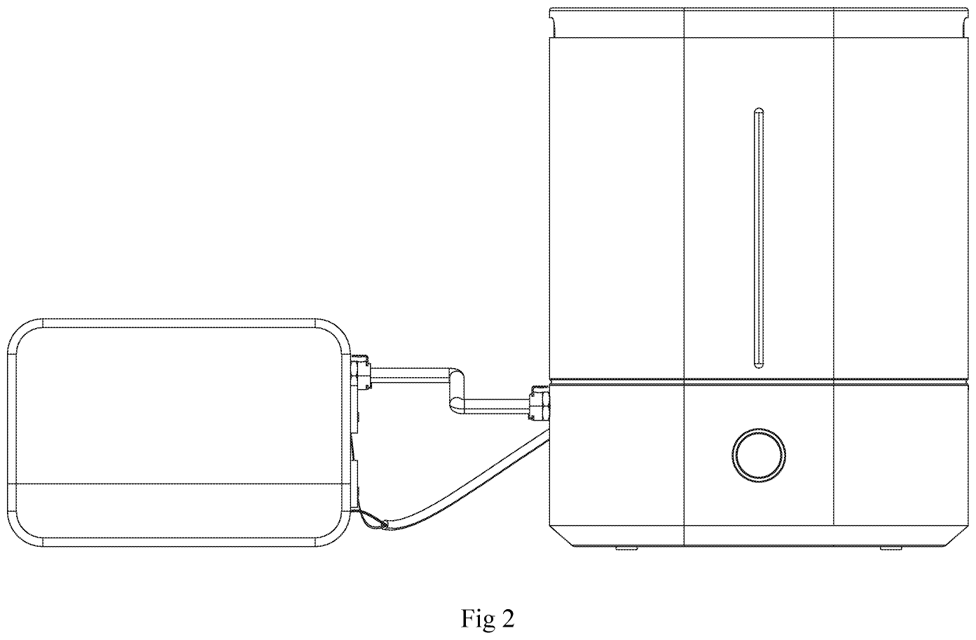

FIG. 3 depicts the inner view of the assembly in accordance with the embodiment of the invention.

FIG. 4 depicts another internal view of the assembly in accordance with the embodiment of the invention.

FIG. 5 depicts the complete exploded view of the assembly in accordance with the embodiment of the invention.

The apparatus and method components have been represented where appropriate by conventional symbols in the drawings, showing only those specific details.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

As discussed above, CPAP water chambers can only hold a few ounces (oz) of water, and the water runs out and dries fast while being used. Therefore, CPAP users have to constantly, and manually refill their CPAP water chambers every night, sometimes more than once per night, because the chamber runs dry and the user must wake up to refill the water chamber. The water in the CPAP water chamber does not last the whole night and this leads to discomfort and issues staying asleep; as well as the unwanted task of constant nightly water replenishing of the CPAP water chamber.

The current invention claimed here solves this problem. The instant invention provides an improved and significant methodology to supply water to automatically refill and replenish a CPAP water chamber for many nights and days. The invention provides clean, fresh, and sanitized water for many days; instead of the CPAP user having only enough water to last for one sleep cycle, a few hours or a much shorter amount of time.

Traditionally the CPAPs water chambers run out of water fast. The water chamber in a CPAP machine typically holds around 11-17 oz of water. During sleep, this water is used to increase the humidity in the air, the CPAP user breaths in; which helps to make breathing and sleeping much easier and more comfortable. Once the water does not last throughout the sleep cycle, the user can experience discomforts, such as dry eyes and throat, nasopharyngeal issues, sleep disruptions and other possible side effects.

The standard water chambers, dry out and run out of water relatively quickly; depending on the humidifier user setting. The claimed invention provides a constant and on-demand supply of fresh, clean sanitized water to the CPAP water chamber; and prevents the user from feeling discomfort associated with a dry water chamber.

The current invention as per its preferred embodiments holds around 3 Liters of water and can supply the CPAP water chamber for many days depending on the CPAP user humidifier settings, for an estimated 7 to 10 days.

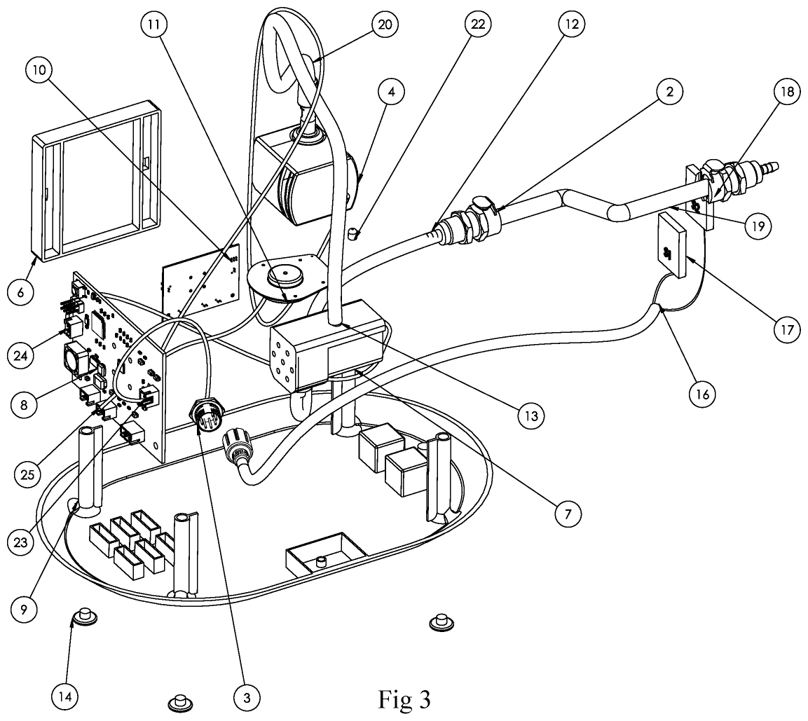

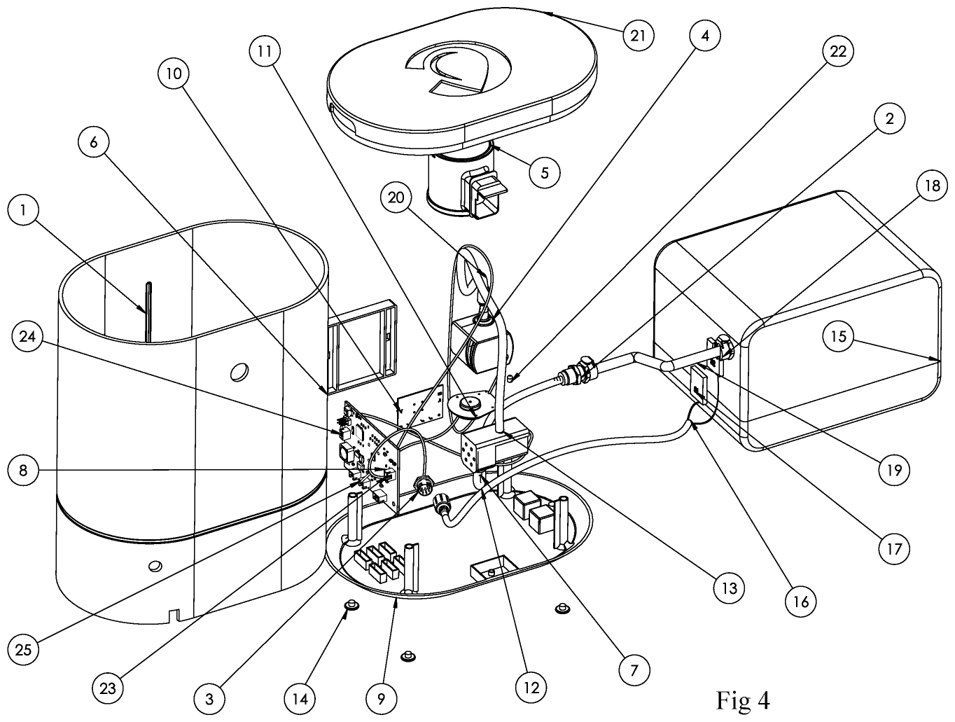

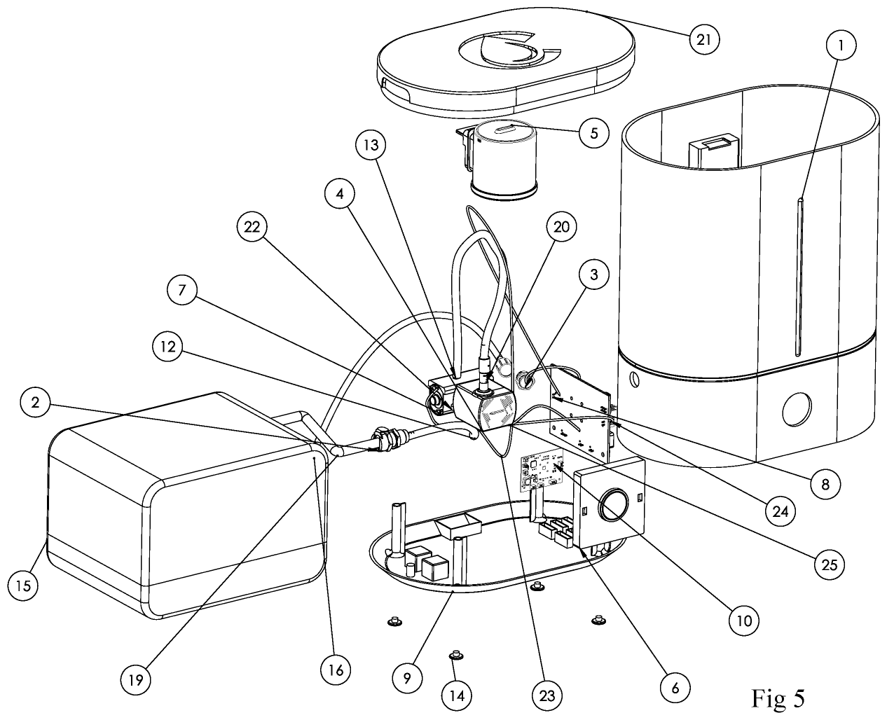

As shown in FIG. 3-5 multiple parts involved in the assembly can be understood. The container (1) holds approximately 3 Liters of water. The CPAP humidifier water chamber (15) has two sensors attached to it (S1 and S2). The water level capacitor sensor-1 S1 (17) starts the submersible water pump (4) that is inside the container (1) when it senses that the water level within the CPAP water chamber (15) is low.

As per further embodiments of the invention, the custom ATO circuit (auto top-off) circuit PCB (8) controls all electronic functions, including the sensors, solenoid and pump, and UVC. It is the brain of the invention. This PCB (circuit) uses firmware to control the logic and programming scheme of for the sensor functions. The logic of the firmware new program is as follows:
1. S2 sensor is used for overflow detection. If the liquid triggers this sensor, the water pump Stops running;
2. The S1 sensor is used for water replenishment detection. If the sensor detects that there is no water, it will start the water pump. The maximum running time is 7 seconds. If it runs overtime, it will be classified as in an “abnormal state” immediately, and the water pump will be stopped at the same time, and the status indicator on the PCB will flash. The abnormal state will be cleared and normal work will resume until sensor S1 detects water again; if there is no timeout and sensor S1 detects water again, the pump will run for 2 more seconds and then stop immediately.

As per further embodiments of the invention, the water is pumped and carried from the container ((1) through the internal and external tubing into the CPAP water chamber (15) this function is carried out when the sensor (S1) detects that there is no water inside the CPAP humidifier water chamber (15). The sensor (S1) triggers the water pump to start pumping water and triggers the solenoid power supply PCB (24) to open and allow water to flow through. The water flows from the container (1), through the submersible water pump (4), up through the internal water transfer hose (13) through the solenoid (24), out of the container through the MCD 1602 male & female quick connect water transfer valves (2), through the external water transfer hose and into the CPAP humidifier water chamber (15).

As per further embodiments of the invention, once the water has filled the CPAP water chamber to the level of Sensor-1 (S1) (17), the sensor detects the presence of water and stops the water pump (4) from running and cuts water supply and at the same time to the solenoid power supply PCB terminal (24) effectively sealing the water transfer hose. This keeps the air pressure produced by the CPAP to enter the hose or tank; maintaining positive therapeutic pressure on the operating CPAP. This process repeats itself automatically as needed while the Invention is in operation.

As per further embodiments of the invention, the anti-overspill water level capacitor sensor-2 (S2) (18) acts as an anti-overflow safety, this sensor will turn off the water pump and trigger a complete stop of power to the water pump, only if and when Sensor-S1 fails. The listed components are made to carry out the previously described functions. Kaomer custom ATO (auto top-off) PCB (8) has specific firmware operational schemes. anti-overfill and spill safety features, like learned water distribution timing. This stops the submersible water pump (4) from pumping after a certain amount of time, in case both sensors (17) and (18) fail. The other kinds of level sensors could be used instead of the non-contact capacitor-based sensors this invention uses.

The assembly working can be understood by the below description. The container (1) holds approximately 3 Liters of water and CPAP humidifier water chamber (15) has two sensors attached to it (S1 and S2). The water level capacitor sensor-1 (S1) (17) starts the submersible water pump (4) water pump that is inside the container (1) once it senses that the water level within the CPAP water chamber (15) is low.

As per further embodiments of the invention, the custom ATO circuit (auto top-off) circuit PCB, controls all electronic functions, including the sensors, solenoid and pump, UVC. It is the brain of the invention. This PCB (circuit) uses firmware to control the logic and programming scheme of for the sensor functions. The logic of the firmware new program is as follows:
1. S2 sensor is used for overflow detection. If the liquid triggers this sensor, the water pump Stops running;
2. The S1 sensor is used for water replenishment detection. If the sensor detects that there is no water, it will start the water pump. The maximum running time is 7 seconds. If it runs overtime, it will be classified as in an “abnormal state” immediately, the water pump will be stopped at the same time, and the status indicator on the PCB will flash. The abnormal state will be cleared and normal work will resume until sensor S1 detects water again; if there is no timeout and sensor S1 detects water again, the pump will run for 2 more seconds and then stop immediately.

As per further embodiments of the invention, the water is pumped and carried from the container (1) through the internal and external tubing into the CPAP water chamber (15) this function is carried out when the sensor (S1) detects that there is no water inside the CPAP humidifier water chamber (15). sensor (S1) triggers the water pump to start pumping water and triggers the solenoid power supply PCB terminal (24) to open and allow water to flow through. The water flows from the container (1), through the submersible water pump (4), up through the internal water transfer hose (13) through the solenoid (24), out of the container through the MCD 1602 male & female quick connect water transfer valves (Item #2), through the external water transfer hose and into the CPAP humidifier water chamber (15).

As per further embodiments of the invention, when the water has filled the CPAP water chamber to the level of the Sensor-1 (S1) (17), the sensor detects the presence of water and stops the water pump (4) from running and cuts water supply and at the same time to the solenoid power supply PCB terminal (24) effectively sealing the water transfer hose. This keeps the air pressure produced by the CPAP entering the hose or tank; maintaining positive therapeutic pressure on the operating CPAP. This process repeats itself automatically as needed while the Invention is in operation.

As per further embodiments of the invention, the anti-overspill water level capacitor sensor-2 (S2) (18) acts as an anti-overflow safety, this sensor will turn off the water pump and trigger a complete stop of power to the water pump, if Sensor-S1 fails.

As per further embodiments of the invention, the listed components are made to carry out the previously described functions. custom ATO (auto top-off) PCB (8) has specific firmware operational schemes. The anti-overfill and spill safety features, like learned water distribution timing. This stops the submersible water pump (4) from pumping after a certain amount of time, in case both sensors (17) and (18) fails. Furthermore, other kinds of level sensors could be used instead of the non-contact capacitor-based sensors this invention uses.

As per further embodiments of the invention, the other components are made to aid and carry out the previously described functions. As well as Connector pins (3) having further anti-overfill and spill safety features, like learned water distribution timing. This stops the Mini submersible water pump (4) from pumping after a certain amount of time, in case both sensors (17) and (18) fail.

As per further embodiments of the invention, the UVC led (11) keeps the water in the container disinfected and inhibits the growth of bacteria and viruses.

As per further embodiments of the invention, the cable harness (16) with dual sensor Y terminals, allows for ease of assembly and disassembly. the sensors as well as the external water tubing can be easily removed if needed.

Skilled artisans will appreciate those elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

1. An automatic CPAP humidifier water chamber replenishing and sanitizing system comprising:
Container (1);
MCD 1602 (2);
GX Connector (3);
Mini Submersible water pump (4);
Top Cylinder (5);
Power switch PCB holder (6);
Solenoid (7);
Custom ATO PCB (8);
The base cover (9);
Multi-step power switch(10);
UVC metal and glass casing (11);
Female screw-on sensor harnesses quick connector (12);
Internal water transfer hose (13);
Rubber feet (14);
CPAP humidifier water chamber (15);
Dual sensor wire terminal (16);
Water level capacitor sensor 1 (17);
Anti-overspill water level capacitor sensor 2 (18);
External water transfer hose (19);
Pump priming valve (20);
Flat lid (21);
LED small (22); and,
Water pump water supply PCB terminal (23).
The automatic CPAP humidifier water chamber replenishing and sanitizing system as per claim 1, wherein the container (1) holds approximately 3 Liters of water.
The automatic CPAP humidifier water chamber replenishing and sanitizing system as per claim 1, wherein the CPAP humidifier water chamber (15) has two sensors attached to it (S1 and S2).
The automatic CPAP humidifier water chamber replenishing and sanitizing system as per claim 1, wherein the water level capacitor sensor-1 S1 (17) starts the submersible water pump (4) that is inside the container (1) once it senses that the water level within the CPAP water chamber (15) is low.
The automatic CPAP humidifier water chamber replenishing and sanitizing system as per claim 1, wherein the custom ATO circuit (auto top-off) circuit PCB (8) controls all electronic functions, including the sensors, solenoid and pump, and UVC.
The automatic CPAP humidifier water chamber replenishing and sanitizing system as per claim 1, wherein the water is pumped and carried from the container ((1) through the internal and external tubing into the CPAP water chamber (15) this function is carried out when the sensor (S1) detects that there is no water inside the CPAP humidifier water chamber (15).
The automatic CPAP humidifier water chamber replenishing and sanitizing system as per claim 1, wherein the sensor (S1) triggers the water pump to start pumping water and triggers the solenoid power supply PCB (24) to open and allow water to flow through.
The automatic CPAP humidifier water chamber replenishing and sanitizing system as per claim 1, wherein the water flows from the container (1), through the submersible water pump (4), up through the internal water transfer hose (13) through the solenoid (24), out of the container through the MCD 1602 male & female quick connect water transfer valves (2), through the external water transfer hose and into the CPAP humidifier water chamber (15).
The automatic CPAP humidifier water chamber replenishing and sanitizing system as per claim 1, wherein once the water has filled the CPAP water chamber to the level of Sensor-1 (S1) (17), the sensor detects the presence of water and stops the water pump (4) from running and cuts water supply and at the same time to the solenoid power supply PCB terminal (24) effectively sealing the water transfer hose.
The automatic CPAP humidifier water chamber replenishing and sanitizing system as per claim 1, wherein the air pressure produced by the CPAP enters the hose or tank; maintaining positive therapeutic pressure on the operating CPAP.
The automatic CPAP humidifier water chamber replenishing and sanitizing system as per claim 1, wherein the anti-overspill water level capacitor sensor-2 (S2) (18) acts as an anti-overflow safety.
The automatic CPAP humidifier water chamber replenishing and sanitizing system as per claim 1, wherein sensor S2 (18) will turn off the water pump and trigger a complete stop of power to the water pump, only if and when Sensor-S1 fails.
The automatic CPAP humidifier water chamber replenishing and sanitizing system as per claim 1, wherein the UVC led (11) keeps the water in the container disinfected and inhibits the growth of bacteria and viruses.
The automatic CPAP humidifier water chamber replenishing and sanitizing system as per claim 1, wherein the cable harness (16) with dual sensor Y terminals, allows for ease of assembly and disassembly. the sensors as well as the external water tubing can be easily removed if needed.

2. The automatic CPAP humidifier water chamber replenishing and sanitizing system including variety of sensors wherein:
As per claim 2, wherein the S1 sensor is used for water replenishment detection.
As per claim 2, wherein the S1 sensor detects that there is no water, it will initiate the water pump.
As per claim 2, wherein the maximum running time is 7 seconds.
As per claim 2, wherein if the water running time exceeds the time limit, it will be classified as in an “abnormal state” immediately, the water pump will be stopped at the same time, and the status indicator on the PCB will flash.
As per claim 2, wherein the abnormal state will be cleared and normal work will resume until sensor S1 detects water again.
As per claim 2, wherein if there is no timeout and sensor S1 detects water again, the pump will run for 2 more seconds and then stop immediately.
As per claim 2, wherein the S2 sensor is used for overflow detection. If the liquid triggers this sensor, the water pump stops running.
